(19) United States
(12) Patent Application Publication
(10) Pub. No.: US 2012/0029347 A1
(43) Pub. Date: Feb. 2, 2012

(54) Method and Device for Treatment Dysphagia

(76) Inventor: Maggie Scott, Galena Park, TX (US)

(21) Appl. No.: 12/848,843

(22) Filed: Aug. 2, 2010

(51) Int. Cl. A61B 5/00

(52) U.S. Cl. 600/431; 600/300

(57) ABSTRACT

Described is a method and products for testing patients having difficulty swallowing specialized capsules. Capsules of various sizes are manufactured. One group of capsules, gels or tablets is manufactured containing s non-active composition and the second group of capsules, gels or tablets are manufactured using an X-Ray imaging agent. The method comprises testing the patient swallowing difficulty utilizing each group of products.

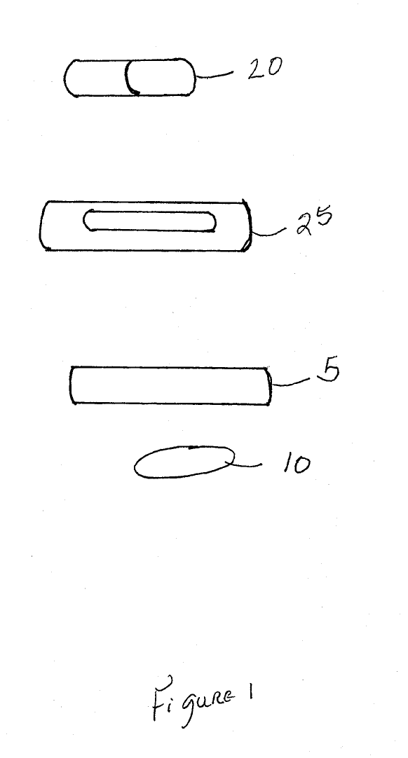

### BACKGROUND OF INVENTION

The present invention relates to a method of treating and evaluating people with swallowing problems. In particular, the present invention relates to a method of treating and evaluating patients having difficulty swallowing tablets and capsules.

Dysphagia is defined normally, as difficulty swallowing. With dysphagia, patients often feel a sensation that suggests a difficulty in the passage of solids or liquids form the mouth into the stomach.

Swallowing problems are generally found in elderly patients, people who have had strokes, or people with head and neck cancer or neurological diseases.

In the prior art there are methods and devices for treating dysphagia disclosed.

However, currently there is no method available for speech pathologists to specifically test and treat for people having problems swallowing their medications.

### SUMMARY OF THE INVENTION

Accordingly, the present invention provides a method and materials for testing and treating patients who may be having difficulty swallowing Swallow (MBS) study which involves providing food and liquid and utilizing X-ray to determine the level of swallow function.

The method of the present invention evaluates or tests the specific problem of swallowing medications which have a different consistency and viscosity then food. Food is normally broken down through the saliva in the mouth. However, medications are specifically coated to prevent the breakdown within the mouth. Thus, patients can have difficulty swallowing capsules or tablets and not food.

The present invention provides a method and product that allows a speech pathologist to evaluate, test and treat swallow function deficits with capsules and tables. Based upon the evaluation utilizing the method and the product of the present invention, a speech pathologist can setup a specific medication plan to suit a particular patient.

Products are broken down into the following types:

Hard capsules (20) which is an encapsulation of a powdered form with or without barium;

Soft gel capsules (25) which is an encapsulation in a gel form;

Tablets (10) is in a powdered pressed compacted solid form; and

Caplets (5) is a coated tablet.

In the present invention the above mentioned products are manufactured in different sizes with and without barium sulfate from extra small, small, medium, large and extra large. The first group of products are manufactured with a non, and the second group is manufactured with barium sulfate.

Different sizes are available to determine which size/type of capsules/tablet is difficult for the person to swallow. Once this is determined a decision can be made whether medications needs to be crushed or changed to ensure safe swallow. Speech pathologist can also utilize the product during therapy sessions to train compensatory strategies for taking medications.

The first group of medications uses a non active composition that is created in a powdered form or in a gel form. The non-active compositions are created in powder form for tablets, hard shell capsules, caplets. Then, a non-active gel type composition is created for the soft gel type capsules. Various sizes of the non-active compositions mentioned above are manufactured for use when X-Ray is not utilized.

The second group of medications uses an imaging agent such as barium sulfate which is the radio contrast agent for X-ray imaging equipment. Barium Sulfate compositions are created in powder form for tablets, hard shell capsules, caplets. Then, a Barium Sulfate Composition in a gel type composition is created for the soft gel type capsules. Various sizes of the product containing barium sulfate are manufactured.

Normally, food cannot be used to test the level of difficulty a patient is having swallowing medications. There is nothing for the doctor to use to test whether or not medications needs to be crushed or provided in liquid form to the patients.

To test the level of difficulty of swallowing, the following steps are performed:
1. performing a bedside physical examination to determine the level of difficulty dealing with (1) labials evaluation which includes evaluation of the tightening of the lips (2) lingual function which includes tongue evaluation (3) laryngeal function which includes voice box evaluation; this testing includes dealing with the sensation of the medications being stuck in throat.
2. based upon the physical examination creating a kit of products consisting of a first group of products and a second group of products;
3. providing a first group of products with a non-active composition;
4. based upon the physical examination, creating at least one subset within the first group of products consisting of tablets, hard shell capsules, soft shell capsules or caplets from extra small to large;
5. providing the patient with a selected subset from the first group of products;
6. starting with the extra small size within the selected subset within the first group of products;
7. allowing the patient to swallow each product within the selected subset in the first group of the products;
8. recording the results of the level of difficulty of swallowing; and
9. repeating steps 5 to 9 for each subset within the first group of products;

To test the next level of difficulty of swallowing, the following steps are performed:
1. providing the patient with a second group of products having an imaging agent such as barium;
2. based upon the physical examination, at least one subset within the second group of products consisting of tablets, hard shell capsules, soft shell capsules or caplets from extra small to large;
3. providing the patient with a selected subset within the second group of products;
4. starting with the extra small size within the at least one subset within the second group of products;
5. allowing the patient to swallow each product within selected subset within the second group of the products;
6. x-raying the patient during the swallowing process of each product within the selected subset to detect the difficulty;
7. recording the results of the level of difficulty of swallowing; and
8. repeating steps 3 to 7 for each subset within the second group of products.

What is claimed is:

1. A kit for a speech pathologist to test or evaluate a patient having swallowing deficits, the kit including a plurality compositions of products selected from the group consisting of
(a) a hard shell capsule containing a non-active composition of a powdered substance manufactured in various sizes;
(b) a soft shell capsule containing a non-active composition of a gel substance manufactured in various sizes;
(c) a tablet containing a non-active composition of a compressed powdered substance manufactured in various sizes;
(d) a non-active caplet containing a non-active composition of a coated compressed powdered substance manufactured in various sizes.

2. The kit of claim 1 wherein the plurality of products are specifically designed based upon a physical examination of the patient having swallowing difficulty.

3. The kit of claim 1 wherein the plurality of products are manufactured based upon a standard physical examination of the patient having swallowing difficulty.

4. The kit according to claim 1 wherein the non-active composition products include an imaging agent.

5. The kit according to claim 4 wherein the imaging agent is a barium sulfate-containing compound.

6. The kit according to claim 1 wherein the hard shell capsule is manufactured from a predetermined extra small size to a predetermined large size.

7. The kit according to claim 1 wherein the soft shell capsule is manufactured from a predetermined extra small size to a predetermined large size.

8. The kit according to claim 1 wherein the tablet is manufactured from a predetermined extra small size to a predetermined large size.

9. The kit according to claim 1 wherein the caplet is manufactured from a predetermined extra small size to a predetermined large size.

10. A method for a speech pathologist to evaluate a patient having swallowing deficits, the method comprising:
(1) providing products selected from the a first group of products consisting of:
(a) a hard shell capsule containing a non-active composition of a powdered substance manufactured in various sizes;
(b) a soft shell capsule containing a non-active composition of a gel substance manufactured in various sizes;
(c) a tablet containing a non-active composition of a compressed powdered substance manufactured in various sizes;
(d) a non-active caplet containing a non-active composition of a coated compressed powdered substance manufactured in various sizes.
(2) based upon a physical examination by the speech pathologist, creating a plurality of subsets of products ranging in size from an extra small predetermined size to an extra large predetermine size within the first group of products;
(3) providing the patient with a selected subset within the first group of products;
(4) starting with the extra small size within the selected subset, allowing the patient to swallow each product in the selected subset within the first group of the products;
(5) as the patient swallows, identifying the level of swallowing difficulty;
(6) recording the results of the level of difficulty of swallowing;
(7) progressively moving up in product size within the selected subset; and
(8) repeating steps (3) through (7) for each subset in the plurality of subsets of products.

11. The method of claim 10 wherein the first group of products contain an imaging agent.

12. The method of claim 11 wherein the imaging agent is Barium Sulfate.
